(19) United States
(12) Patent Application Publication
(10) Pub. No.: US 2008/0269500 A1
(43) Pub. Date: Oct. 30, 2008

(54) Process for the Preparation of 4-Aminopyrazole Derivatives

(75) Inventor: George Beresford Hill, Cheshire (GB)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/067,253

(22) PCT Filed: Sep. 20, 2006
(86) PCT No.: PCT/GB2006/003500

§ 371 Date: Mar. 18, 2008

Related U.S. Application Data
(60) Provisional application No. 60/719,437, filed on Sep. 22, 2005.
(60) Provisional application No. 60/731,397, filed on Oct. 28, 2005.

(51) Int. Cl. C07D 231/38; C07C 233/01

(52) U.S. Cl. 548/371.7; 564/193; 548/372.5

(57) ABSTRACT

Processes for the preparation of 4-aminopyrazole and derivatives thereof, such as those of formula (C), which are useful as intermediates in the preparation of pharmaceutical compounds; to certain compounds used in these processes; and to processes for the preparation of said compounds.

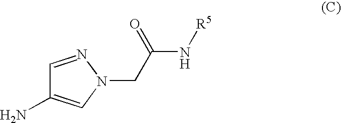

The present invention relates to a process for the preparation of 4-aminopyrazole derivatives, which are useful as intermediates in the preparation of pharmaceutical compounds, to certain compounds used in this process and to processes for the preparation of said compounds.

Acetanilide substituted pyrazole-aminoquinazoline compounds are known to inhibit one or more of the Aurora kinases, serine-threonine protein kinases which have been implicated in human hyperproliferative disease (Adams et al., 2001, Trends in Cell Biology. 11(2): 49-54; Bischoff et al., 1998, The EMBO Journal. 17(11): 3052-3065; Adams et al., 2001, Chromsoma. 110(2):65-74; and Kimura et al., 1999, Journal of Biological Chemistry. 274(11): 7334-40). In particular, International Patent Application No. PCT/GB04/01614 (Publication No. WO04/94410) discloses that compounds of formula (A) are useful in the treatment of hyperproliferative disease such as cancer:

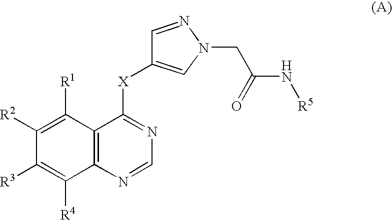

The definitions for X, R¹, R², R³, R⁴ and R⁵ provided in WO04/94410 are incorporated herein by reference, but in particular X is —O—, —NH— or —N(C₁₋₄alkyl)- and R⁵ is optionally substituted aryl or heteroaryl. Specific examples of such compounds are N-(3-fluorophenyl)-2-{4-[(6-methoxy-7-{3-[methyl(propyl)amino]propoxy}quinazolin-4-yl)amino]-1H-pyrazol-1-yl}acetamide, N-(2,3-difluorophenyl)-2-[4-({7-methoxy-5-[(2R)-pyrrolidin-2-ylmethoxy]quinazolin-4-yl}amino)-1H-pyrazol-1-yl]acetamide and 2-(4-{[7-(3-chloropropoxy)quinazolin-4-yl]amino}-1H-pyrazol-1-yl)-N-(2,3-difluorophenyl)acetamide. These compounds may be prepared by a method which involves the reaction of a compound of formula (B):

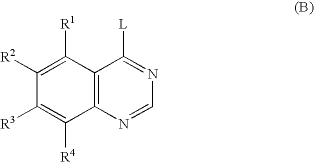

where L is a suitable leaving group such as chloro, bromo, SMe etc. and R¹, R², R³ and R⁴ are as defined in WO04/94410 and are incorporated herein by reference, with a compound of formula (C):

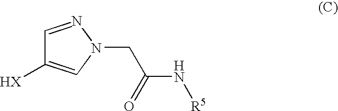

where X and R⁵ are as defined in WO04/94410 and are incorporated herein by reference. This reaction is generally performed in the presence of hydrochloric acid in dioxane under an inert atmosphere.

The compound of formula (C) where X is NH can be prepared in a two step process which involves the coupling of (4-nitro-1H-pyrazol-1-yl)acetic acid (E) with R⁵NH₂ followed by reduction of the resulting (4-nitro-1H-pyrazol-1-yl)acetamide derivative (D) as shown in scheme 1:

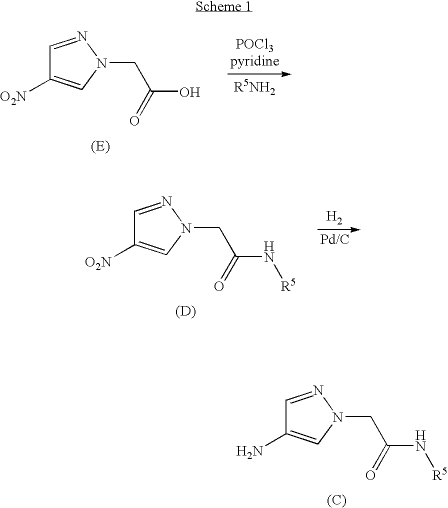

(4-nitro-1H-pyrazol-1-yl)acetic acid (E) is accessed via 4-nitropyrazole which in turn is derived from 1-nitropyrazole by acidic rearrangement. Unfortunately 1-nitropyrazole has explosive properties so this route to a compound of formula (C) is inappropriate for use in a large-scale manufacturing process. Another known route to 4-nitropyrazole uses sodium nitromalonaldehyde but this reagent also has explosive properties. 4-aminopyrazole and derivatives thereof have been prepared using a perchlorate salt but this is another reagent that is likely to suffer from thermally instability (Valiullin V. A., Ivakhnenko T. E., Doklady Chemistry 2004, 399, 214). An alternative route to a compound of formula (C) is thus required, which does not involve the use of explosive reagents.

Dousson et al. (Dousson C. B., Heron N. M., Hill G. B., Synthesis. 2005, No. 11, 1817-1821) have previously avoided the use of hazardous precursors in the synthesis of 2-functionalised 5-aminopyrimidines by condensing a vinamidinium dihexafluorophosphate salt with functionalised amidines. Pyrazoles with nitrogen linked heterocyclic substituents in the 4-position have also been prepared from vinamidinium salts (Adams F., Gompper R., Kujath E., Angewandte Chemie. 1989, 101, 1043; and Gupton J. T., Hicks F. A., Smith S. Q., Main A. D., Petrich S. A., Wilkinson D. R., Sikorski J. A., Katritzky A. R., Tetrahedron. 1993, 49, 10205). An alternative method to 4-aminopyrazole derivatives wherein the 4-amino group is unsubstituted is still required. Fortunately, we have been able to provide a process for the preparation of 4-aminopyrazole derivatives of formula (C) which does not involve the use of explosive reagents such as 1-nitropyrazole, sodium nitromalonaldehyde or perchlorate salts.

We have discovered that a compound of formula (C) can be prepared according to scheme 2:

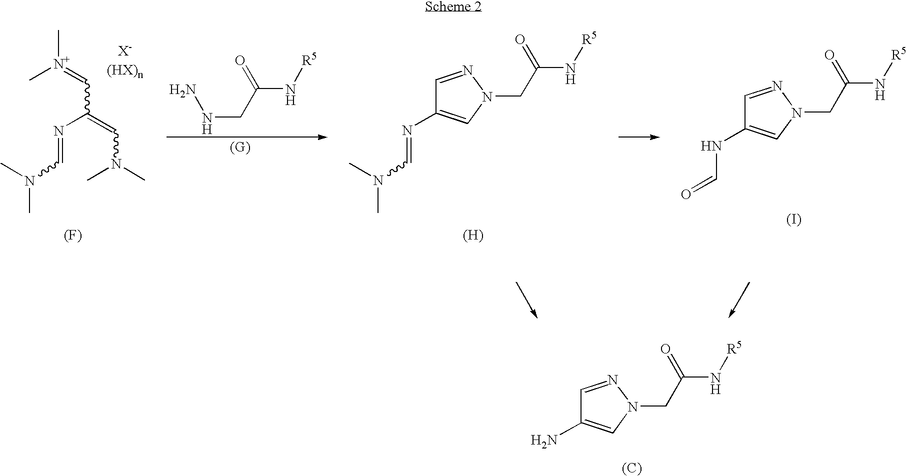

wherein X is PF₆ or BF₄; n is 0 or 1; and R⁵ is optionally substituted aryl or heteroaryl.

Accordingly, the present invention provides a process comprising the reaction of a compound of formula (G)

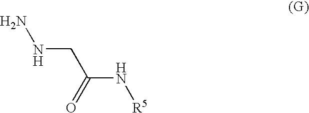

with a compound of formula (F)

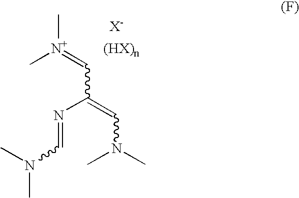

in the presence of a base; wherein X is PF₆ or BF₄; n is 0 or 1; and R⁵ is optionally substituted aryl or heteroaryl, such as aryl or heteroaryl optionally substituted by 1, 2 or 3 substituents independently selected from halo, hydroxy, cyano, nitro, amino, C₁₋₄alkylamino, di(C₁₋₄alkyl)amino, C₁₋₄alkyl, C₂₋₄alkenyl, C₂₋₄alkynyl, C₁₋₄alkoxy, —C(O)NH₂, —C(O)NHC₁₋₄alkyl, —C(O)NHC₃₋₆cycloalkyl, —C(O)NHC₂₋₄alkenyl, —C(O)NHC₂₋₄alkynyl, —NHC(O)H, —NHC(O)C₁₋₄alkyl, —NHC(O)C₃₋₆cycloalkyl, —NHC(O)C₂₋₄alkenyl, —NHC(O)C₂₋₄alkynyl, —S(O)ₚH, —S(O)ₚC₁₋₄alkyl, —S(O)ₚC₃₋₆cycloalkyl, —S(O)ₚC₂₋₄alkenyl and —S(O)ₚC₂₋₄alkynyl where p is 0, 1 or 2.

Preferably, this reaction is performed in the presence of a base such as sodium methoxide, sodium ethoxide, N,N-diisopropylethylamine or potassium tert-butoxide in organic solvents such as pyridine, methanol, ethanol, acetonitrile or chloroform. The reaction may also be performed in the presence of sodium hydroxide in chloroform, aqueous dioxane, aqueous dimethylformamide or dimethylacetamide. In particular the reaction is performed in the presence of sodium methoxide in pyridine. The base may be present in a catalytic amount but preferably one stoichometric equivalent of the base is used.

It is preferred that the reaction be performed at a temperature in the range of −40° C. to +75° C. A more preferred temperature is in the range −40° C. to +20° C. Low temperatures such as those in the range of −15° C. to −30° C. are more preferred so that the formation of by-products is minimised. Even more preferably, the reaction is performed at approximately −30° C.

It is also advantageous to use the purified stoichometric salts of the compound of formula (G) and the compound of formula (F) to achieve good yields.

Certain of compounds of formula (G) as defined herein form further aspects of the invention; for example when R⁵ is aryl or heteroaryl and particularly when R⁵ is aryl, or when R⁵ is aryl or heteroaryl substituted by 1 or 2 halo. In preferred compounds of formula (G), R⁵ is aryl substituted by 1 or 2 halo and particularly phenyl substituted by 1 or 2 chloro or fluoro and more particularly fluoro. For example, {2-[(2,3-difluorophenyl)amino]-2-oxoethyl}hydrazine and {2-[(3-fluorophenyl)amino]-2-oxoethyl}hydrazine and salts thereof such as the methanesulfonate salts are particularly interesting compounds.

The compound of formula (G) can be prepared by reacting a compound of formula (J)

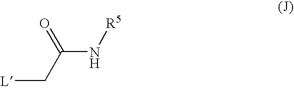

wherein L′ is a leaving group such as halo, mesyl or tosyl; with hydrazine such as the hydrate, a hydrochloride salt or suitably protected hydrazine in a suitable organic solvent such as methanol or ethanol, and deprotecting if required. Preferably L′ is halo such as bromo or chloro and more preferably bromo. The reaction may be performed in a neutral or basic solution such as in the presence of potassium hydrogen carbonate or potassium carbonate and in a solvent such as ethyl acetate or acetonitrile. A suitable protecting group for hydrazine is the tert-butoxycarbonyl protecting group or the benzyloxycarbamate protecting group. Removal of this protecting group is effected using conventional methods such as those described in Protective Groups in Organic Synthesis, 2^{nd} Edition, by Green et al., published by John Wiley & Sons. Preferably L′ is bromo wherein the compound of formula (J) may be prepared by reacting R⁵NH₂ with 2-bromoacetyl bromide in the presence of a base such as sodium hydroxide and in a solvent such as diethyl ether.

The reaction of a compound of formula (G) with a compound of formula (F) yields a compound of formula (H) wherein R⁵ is as defined herein:

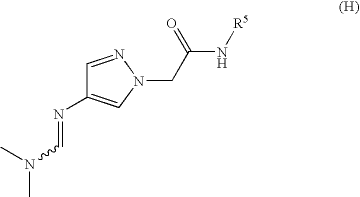

This compound is a novel intermediate and forms a further aspect of the invention.

A compound of formula (H) may be hydrolysed to yield a compound of formula (I) wherein R⁵ is as defined herein:

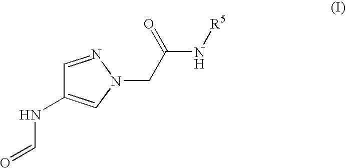

Hydrolysis is suitably performed by treatment with a basic solution such as aqueous ammonia in water or n-propanol. Alternatively acidic conditions may be used, for example by using mineral acids, buffered solutions or alkanoic acids with or without one or more co-solvents. Examples of mineral acids are hydrochloric acid and sulfuric acid. Buffered solutions have acidic pH values, preferably in the range of pH 3 to 4 and a preferred buffered solution is a phosphate buffer. Examples of alkanoic acids include acetic acid and propanoic acid. The choice of co-solvent will depend on the mineral acid, buffer solution or alkanoic acid chosen but suitable co-solvents will be known to the skilled person. Particular examples of co-solvents are ethanol and tetrahydrofuran. Hydrolysis may be performed by treatment with aqueous potassium carbonate in an organic solvent such as dioxane, anhydrous zinc chloride in an organic solvent such as ethanol, aqueous zinc chloride, or aqueous sulfuric acid. This conversion may be carried out at a range of temperature but can be conveniently performed at ambient temperature or under reflux conditions.

The compound of formula (I) is a novel intermediate and forms a further aspect of the invention.

Also provided is a process for the preparation of a compound of formula (C) from a compound of formula (I) which process comprises deformylation of the compound of formula (I). Deformylation may be performed by using acidic conditions for example by using mineral acids, buffered solutions or alkanoic acids with or without one or more co-solvents. Examples of mineral acids are hydrochloric acid and sulfuric acid. Buffered solutions have acidic pH values, preferably in the range of pH 3 to 4 and a preferred buffered solution is a phosphate buffer. Examples of alkanoic acids include acetic acid and propanoic acid. The choice of co-solvent will depend on the mineral acid, buffer solution or alkanoic acid chosen but suitable co-solvents will be known to the skilled person. Particular examples of co-solvents are ethanol and tetrahydrofuran. It is particularly preferred to use aqueous sulfuric acid. Alternatively deformylation may be effected with aqueous potassium carbonate in an organic solvent such as dioxane, anhydrous zinc chloride in an organic solvent such as ethanol or aqueous zinc chloride. This conversion may be carried out at a range of temperature but can be conveniently performed at ambient temperature or under reflux conditions.

Alternatively, a compound of formula (C) can be derived directly from a compound of formula (H). This conversion is effected under acidic conditions. Mineral acids, buffered is solutions or alkanoic acids may be used with or without one or more co-solvents. Examples of mineral acids are hydrochloric acid and sulphuric acid. Buffered solutions have acidic pH values, preferably in the range of pH 3 to 4 and a preferred buffered solution is a phosphate buffer. Examples of alkanoic acids include acetic acid and propanoic acid. The choice of co-solvent will depend on the mineral acid, buffer solution or alkanoic acid chosen but suitable co-solvents will be known to the skilled person. Particular examples of co-solvents are ethanol and tetrahydrofuran. This conversion may be carried out at a range of temperature but can be conveniently performed at ambient temperature or under reflux conditions.

A process for the preparation of a compound of formula (C) thus comprises the steps of:
1. reacting a compound of formula (G) with a compound of formula (F) to yield a compound of formula (H);
2. hydrolysing the compound of formula (H) to yield a compound of formula (I); followed by
3. deformylation of the compound of formula (I).

The reactions described in steps 2. and 3. above may be performed as separate sequential reaction steps where a compound of formula (I) is isolated or they may be performed as a one pot reaction, i.e. without isolating the compound of formula (I). In the latter case, the reagents provided herein for each step should be added to the reaction mixture sequentially. Preferred reagents for this latter case include aqueous potassium carbonate in an organic solvent such as dioxane, anhydrous zinc chloride in an organic solvent such as ethanol or aqueous zinc chloride. Alternatively steps 2. and 3. may be replaced with step 2′ wherein a compound of formula (H) is directly converted to a compound of formula (C).

For the avoidance of doubt, the preferred reaction conditions and reagents described herein in relation to each of these reactions may be incorporated into one or more of steps 1, 2, 3 and 3′ as appropriate.

With regard to a compound of formula (F) and all aspect and embodiments of the invention relating to this compound, X may be PF₆ wherein n is 0 or 1. Preferably X is PF₆ and n is 0. Additionally X may be BF₄ wherein n is 0 or 1 and preferably when n is 1.

In all aspect and embodiments of the invention where R⁵ is optionally substituted aryl or heteroaryl, R⁵ may be aryl or heteroaryl optionally substituted by 1, 2 or 3 substituents independently selected from halo, hydroxy, cyano, nitro, amino, C₁₋₄alkylamino, di(C₁₋₄alkyl)amino, C₁₋₄alkyl, C₂₋₄alkenyl, C₂₋₄alkynyl, C₁₋₄alkoxy, —C(O)NH₂, —C(O)NHC₁₋₄alkyl, —C(O)NHC₃₋₆cycloalkyl, —C(O)NHC₂₋₄alkenyl, —C(O)NHC₂₋₄alkynyl, —NHC(O)H, —NHC(O)C₁₋₄alkyl, —NHC(O)C₃₋₆cycloalkyl, —NHC(O)C₂₋₄alkenyl, —NHC(O)C₂₋₄alkynyl, —S(O)ₚH, —S(O)ₚC₁₋₄alkyl, —S(O)ₚC₃₋₆cycloalkyl, —S(O)ₚC₂₋₄alkenyl and —S(O)ₚC₂₋₄alkynyl where p is 0, 1 or 2. Further, in all aspect and embodiments of the invention R⁵ may be aryl optionally substituted by 1 or 2 halo. Alternatively R⁵ is phenyl optionally substituted by 1 or 2 fluoro or chloro. R⁵ may also be phenyl optionally substituted by 1 or 2 fluoro. In particular R⁵ is 2,3-difluorophenyl, 3-fluorophenyl, 2-fluorophenyl or 2,6-difluorophenyl and more particularly R⁵ is 2,3-difluorophenyl or 3-fluorophenyl.

We have further discovered that a compound of formula (K) can be prepared from a compound of formula (F):

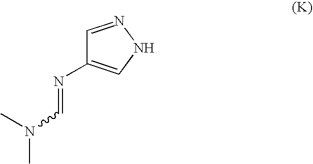

Accordingly, a further process is provided comprising the reaction of a compound of formula (F)

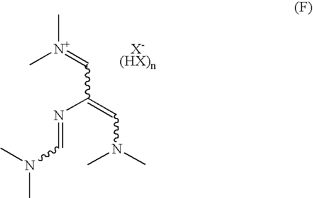

with hydrazine; wherein X is PF₆ or BF₄ and n is 0 or 1. Hydrazine may be used in the form of an anhydrous, a hydrochloride salt such as the mono hydrochloride salt or when suitably protected.

Preferably, this reaction is performed in the presence of a base such as sodium methoxide, sodium ethoxide, N,N-diisopropylethylamine or potassium tert-butoxide in organic solvents such as pyridine, methanol, ethanol, acetonitrile or chloroform. The reaction may also be performed in the presence of sodium hydroxide in chloroform, aqueous dioxane, aqueous dimethylformamide or dimethylacetamide. In particular the reaction is performed in the presence of sodium methoxide in pyridine. The base may be present in a catalytic amount but preferably one stoichometric equivalent of the base is used.

It is preferred that the reaction be performed at a temperature in the range of −40° C. to +75° C. A more preferred temperature is in the range −40 to +20° C. Low temperatures such as those in the range of −15° C. to −30° C. are more preferred so that the formation of by-products is minimised. Even more preferably, the reaction is performed at approximately −30° C. It is also advantageous to use the purified stoichometric salts of hydrazine and the compound of formula (F) to achieve good yields.

The reaction of a compound of formula (F) with hydrazine yields a compound of formula (K) as defined herein. This compound is a novel intermediate and forms a further aspect of the invention. Preferably, this reaction is performed in the presence of a base such as sodium methoxide, sodium ethoxide, N,N-diisopropylethylamine or potassium tert-butoxide in organic solvents such as pyridine, methanol, ethanol, acetonitrile or chloroform. The reaction may also be performed in the presence of sodium hydroxide in chloroform, aqueous dioxane, aqueous dimethylformamide or dimethylacetamide. In particular the reaction is performed in the presence of sodium methoxide in methanol.

The compound of formula (K) can be converted into a compound of formula (H) as defined herein by reacting it with a compound of formula (J) as defined herein. Preferably this reaction is performed in the presence of a base such as sodium methoxide, potassium carbonate or sodium hydride in an organic solvent such as methanol, dimethylformamide or 1,4-dioxane. More preferably the reaction is performed in the presence of a base such as potassium carbonate or sodium hydride in an organic solvent such as dimethylformamide or 1,4-dioxane.

The compound of formula (H) may then be converted into a compound of formula (C) as described herein.

A compound of formula (K) may also be hydrolysed to yield a compound of formula (L)

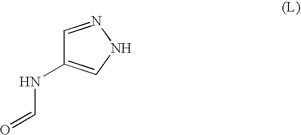

Hydrolysis is suitably performed by treatment with a basic solution such as aqueous ammonia in water or n-propanol. Alternatively acidic conditions may be used, for example by using mineral acids, buffered solutions or alkanoic acids with or without one or more co-solvents. Examples of mineral acids are hydrochloric acid and sulfuric acid. Buffered solutions have acidic pH values, preferably in the range of pH 3 to 4 and a preferred buffered solution is a phosphate buffer. Examples of alkanoic acids include acetic acid and propanoic acid. The choice of co-solvent will depend on the mineral acid, buffer solution or alkanoic acid chosen but suitable co-solvents will be known to the skilled person. Particular examples of co-solvents are ethanol and tetrahydrofuran. Hydrolysis may be performed by treatment with aqueous potassium carbonate in an organic solvent such as dioxane, anhydrous zinc chloride in an organic solvent such as ethanol, aqueous zinc chloride, or aqueous sulfuric acid. This conversion may be carried out at a range of temperature but can be conveniently performed at ambient temperature or under reflux conditions.

The compound of formula (L) is novel and forms a further aspect of the invention.

Deformylation of a compound of formula (L) yields 4-amino pyrazole. Deformylation may be performed by using acidic conditions, for example by using mineral acids, buffered solutions or alkanoic acids with or without one or more co-solvents. Examples of mineral acids are hydrochloric acid and sulfuric acid. Buffered solutions have acidic pH values, preferably in the range of pH 3 to 4 and a preferred buffered solution is a phosphate buffer. Examples of alkanoic acids include acetic acid and propanoic acid. The choice of co-solvent will depend on the mineral acid, buffer solution or alkanoic acid chosen but suitable co-solvents will be known to the skilled person. Particular examples of co-solvents are ethanol and tetrahydrofuran. Preferably aqueous sulfuric acid is used. Alternatively deformylation may be effected with aqueous potassium carbonate in an organic solvent such as dioxane, anhydrous zinc chloride in an organic solvent such as ethanol or aqueous zinc chloride. This conversion may be carried out at a range of temperature but can be conveniently performed at ambient temperature or under reflux conditions.

Alternatively a compound of formula (K) may be converted directly to 4-aminopyrazole under acidic conditions, for example by using mineral acids, buffered solutions or alkanoic acids with or without one or more co-solvents. Examples of mineral acids are hydrochloric acid and sulfuric acid. Buffered solutions have acidic pH values, preferably in the range of pH 3 to 4 and a preferred buffered solution is a phosphate buffer. Examples of alkanoic acids include acetic acid and propanoic acid. The choice of co-solvent will depend on the mineral acid, buffer solution or alkanoic acid chosen but suitable co-solvents will be known to the skilled person. Particular examples of co-solvents are ethanol and tetrahydrofuran. It is particularly preferred to use aqueous sulfuric acid. This conversion may be carried out at a range of temperature but can be conveniently performed at ambient temperature or under reflux conditions.

In this specification the term alkyl when used either alone or as a suffix or prefix includes straight-chain and branched-chain saturated structures comprising carbon and hydrogen atoms. References to individual alkyl groups such as propyl are specific for the straight-chain version only and references to individual branched-chain alkyl groups such as tert-butyl are specific for the branched chain version only. An analogous convention applies to other generic terms such as alkenyl and alkynyl. Examples of C₁₋₄alkyl include methyl, ethyl, propyl, isopropyl, butyl, isobutyl and tert-butyl, examples of C₂₋₄alkenyl include vinyl, allyl and but-2-enyl and examples of C₂₋₄alkynyl include ethynyl, propargyl and prop-1-ynyl.

Cycloalkyl is a monocyclic alkyl group. Examples of for C₃₋₆cycloalkyl include cyclopropyl, cyclobutyl, cyclopentyl and cyclohexyl.

The prefix Cₘ₋ₙ in Cₘ₋ₙalkyl and other terms (where m and n are integers) indicates the range of carbon atoms that are present in the group, for example C₁₋₃alkyl includes C₁alkyl (methyl), C₂alkyl (ethyl) and C₃alkyl (propyl or isopropyl).

The term halo includes fluoro, chloro, bromo and iodo.

Aryl groups are aromatic carbocyclic rings which may be monocyclic or bicyclic. In particular aryl is phenyl or naphthyl.

Unless otherwise stated heteroaryl groups are monocyclic or bicyclic aromatic rings containing 5 to 10 ring atoms of which 1, 2, 3 or 4 ring atoms are chosen from nitrogen, sulfur or oxygen where a ring nitrogen or sulfur may be oxidised. In particular heteroaryl includes furyl, thienyl, pyrrolyl, pyrazolyl, pyridyl, pyrazinyl, pyridazinyl, pyrimidinyl, quinazolinyl and quinolinyl.

This specification also makes use of several composite terms to describe groups comprising more than one functionality. Such terms are to be interpreted as is understood in the art. For example Cₘ₋ₙalkylamino comprises amino substituted by Cₘ₋ₙalkyl whilst —NHC(O)C₃₋₆cycloalkyl comprises a —NHC(O)R functionality bonded through nitrogen wherein R is C₃₋₆cycloalkyl.

Where optional substituents are chosen from 1 or 2 or from 1, 2, or 3 groups or substituents it is to be understood that this definition includes all substituents being chosen from one of the specified groups i.e. all substituents being the same or the substituents being chosen from two or more of the specified groups i.e. the substituents not being the same.

Unless specifically stated the bonding atom of a group may be any atom of that group so for example propyl includes prop-1-yl and prop-2-yl.

Compounds of the present invention have been named with the aid of computer software (ACD/Name version 8.0).

Within the present invention, it is to be understood that, insofar as certain of compounds as defined herein may exist in optically active or racemic forms by virtue of one or more asymmetric carbon or sulfur atoms, the invention includes in its definition any such optically active or racemic form. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to herein.

Within the present invention it is to be understood that a compound as defined herein may exhibit the phenomenon of tautomerism and that the formulae drawings within this specification can represent only one of the possible tautomeric forms. It is to be understood that the invention encompasses any tautomeric form and is not to be limited merely to any one tautomeric form utilised within the formulae drawings.

The compounds described herein may also be present as salts thereof. Salts may, for example, include acid addition salts of compounds of the invention as herein defined which are sufficiently basic to form such salts. Such acid addition salts include but are not limited to furmarate, methanesulphonate, hydrochloride, hydrobromide, citrate and maleate salts and salts formed with phosphoric and sulphuric acid. In addition where compounds of the invention are sufficiently acidic, salts are base salts and examples include but are not limited to, an alkali metal salt for example sodium or potassium, an alkaline earth metal salt for example calcium or magnesium, or organic amine salt for example triethylamine, ethanolamine, diethanolamine, triethanolamine, morpholine, N-methylpiperidine, N-ethylpiperidine, dibenzylamine or amino acids such as lysine.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron trifluoride or boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

### EXAMPLES

The following examples illustrate the invention, in which standard techniques known to the skilled chemist and techniques analogous to those described in these. In the examples, unless otherwise stated:

(i) evaporations were carried out by rotary evaporation in vacuo and work up procedures were carried out after removal of residual solids such as drying agents by filtration;
(ii) operations were carried out at ambient temperature, typically in the range 18-25° C. and in air unless stated, or unless the skilled person would otherwise operate under an atmosphere of an inert gas such as argon;
(iii) column chromatography (by the flash procedure) and medium pressure liquid chromatography (MPLC) were performed on Merck Kieselgel silica (Art. 9385);
(iv) yields are given for illustration only and are not necessarily the maximum attainable;
(v) the structures of the end products of the formula (I) were generally confirmed by nuclear (generally proton) magnetic resonance (NMR) and mass spectral techniques; proton magnetic resonance chemical shift values were measured in deuterated dimethyl sulphoxide (DMSO d₆) (unless otherwise stated) on the delta scale (ppm downfield from tetramethylsilane) using one of the following four instruments
Varian Gemini 2000 spectrometer operating at a field strength of 300 MHz
Bruker DPX300 spectrometer operating at a field strength of 300 MHz
JEOL EX 400 spectrometer operating at a field strength of 400 MHz
Bruker Avance 500 spectrometer operating at a field strength of 500 MHz
   Peak multiplicities are shown as follows: s, singlet; d, doublet; dd, double doublet; t, triplet; q, quartet; qu, quintet; m, multiplet; br s, broad singlet.
   (ix) Preparative high performance liquid chromatography (HPLC) was performed on either

Waters preparative LCMS instrument, with retention time (RT) measured in minutes:

Column: β-basic Hypercil (21×100 mm) 5 μm
Solvent A: Water/0.1% Ammonium carbonate

### Solvent B: Acetonitrile

Flow rate: 25 ml/min
Run time: 10 minutes with a 7.5 minute gradient from 0-100% B
Wavelength: 254 nm, bandwidth 10 nm
Injection volume 1-1.5 ml
Mass detector: Micromass ZMD

Gilson preparative HPLC instrument, with retention time (RT) measured in minutes:

Column: 21 mm×15 cm Phenomenex Luna2 C18
Solvent A: Water+0.2% trifluoracetic acid,
Solvent B: Acetonitrile+0.2% trifluoracetic acid
Flow rate: 21 ml/min
Run time: 20 minutes with various 10 minute gradients from 5-100% B
Wavelength: 254 nm, bandwidth 10 nm
Injection volume 0.1-4.0 ml
(vi) intermediates were not generally fully characterised and purity was assessed by thin layer chromatography (TLC), HPLC, infra-red (IR), MS or NMR analysis.

The process of the invention has been used in the preparation of 2-(4-{[7-(3-chloropropoxy)-quinazolin-4-yl]amino}-1H-pyrazol-1-yl)-N-(2,3-difluorophenyl)acetamide hydrochloride

### N-(3-(Dimethylamino)-2-{[(dimethylamino)methylene]amino}prop-2-en-1-ylidene)-N-methylmethanaminium hydrogen di-hexafluorophosphate

Phosphorus oxychloride (70 ml, 0.75 mol) was added dropwise to dimethylformamide (150 ml) at 10° C., and the mixture was then stirred for 20 minutes at 20° C. This solution was cooled to 5° C. and powdered glycine hydrochloride (27.9 g, 0.25 mol) was added in portions; the temperature of the reaction mixture was maintained at 20° C. The mixture was then heated to 80±2° C. (internal temperature). The solid rapidly disappeared and there was slight effervescence. After 4 hours, the still hot, dark brown solution was poured directly in a fine stream into water (400 ml), pre-cooled to 5° C., the temperature was kept below 20° C. with a dry ice/isopropanol bath. After stirring the solution for 5 minutes, it was cooled to −5° C. and treated from a plastic vessel with 60% aqueous hexafluorophosphoric acid (74 ml, 0.5 mol). A thick precipitate formed immediately and was filtered off, washed with ethanol (500 ml) and air-dried to constant weight over 2 hours. In subsequent runs the product contained up to 0.2 extra equivalents of hexafluorophosphoric acid.

Yield: 73.1 g (60%); off-white solid, mp 165-185° C. decomp.

1H NMR (DMSO-d6): δ=10.15 (d, J=12 Hz, 1H); 8.05 (d, J=12 Hz, 1H); 7.67 (s, 2H); 3.27 (m, 9H); 3.15 (m, 9H).

13C NMR (DMSO-d6): δ=161.11, 158.53, 100.85, 49.17, 43.96, 40 (under DMSO), 36.97.

MS: m/z=197 (M+).

Anal. Calcd. for C₁₀H₂₂N₄.PF₆.1.2HPF₆: C, 23.2; H, 4.29; N, 10.82. Found: C, 23.46; H, 4.26; N, 10.60.

### N-(3-(Dimethylamino)-2-{[(dimethylamino)methylene]amino}prop-2-en-1-ylidene)-N-methylmethanaminium hydrogen mono-hexafluorophosphate

A suspension of crude N-(3-(dimethylamino)-2-{[(dimethylamino)methylene]amino}prop-2-en-1-ylidene)-N-methylmethanaminium hydrogen di-hexafluorophosphate (10 g, 19.3 mmol) in ethanol (80 ml) was treated with triethylamine (8 ml, 58 mmol) and heated to 70° C. giving a clear solution which was cooled immediately. The solution was cooled to −20° C. and the heavy, cream-coloured solid was filtered off, washed with very cold ethanol and ether, and air-dried under a nitrogen blanket.

Yield: 6.6 g (94%).

1H NMR (DMSO-d6): δ=7.33 (s, 2H); 3.16 (s, 12H); 2.95 (s, 3H); 2.89 (s, 3H).

MS: m/z=197 (M+).

Anal. Calcd. for C₁₀H₂₁N₄ PF₆: C, 35.09; H, 6.18; N, 16.37. Found: C, 35.1; H, 6.17; N, 16.1.

### 2-Bromo-N-(2,3-difluorophenyl)acetamide

A solution of 2,3-difluoroaniline (12.9 g, 100 mmol) in diethyl ether (100 ml) was treated with 1M aq NaOH (98 ml, 98 mmol) and stirred vigorously while a solution of bromoacetyl bromide (23.7 g, 117 mmol) in diethyl ether (500 ml) was added dropwise over 20 minutes at 5° C. The mixture was allowed to warm to 20° C. over 1 hour, the layers were separated and the organic phase was washed with 20% aq KHCO₃ (50 ml), dried and concentrated to give a white solid which was dissolved in hot tetrahydrofuran (30 ml), diluted with cyclohexane (300 ml) and isohexane (100 ml), evaporated to 250 ml and cooled. The product, 2-bromo-N-(2,3-difluorophenyl)acetamide, was filtered off.

Yield: 22.69 g, (91%); shining white plates.

¹H-NMR (DMSO-d6): δ=10.34 (br s, 1H), 7.67 (m, 1H), 7.18 (m, 2H), 4.14 (s, 2H).

MS: m/z=250 (M⁻).

Anal. Calcd. for C₈H₆BrF₂NO: C, 38.43; H, 2.42; N, 5.60. Found: C, 38.3; H, 2.27; N, 5.37.

### Tert-butyl 2-{2-[(2,3-difluorophenyl)amino]-2-oxoethyl}hydrazinecarboxylate

A solution of 2-bromo-N-(2,3-difluorophenyl)acetamide (20 g, 80 mmol) in ethyl acetate (100 ml) was treated with 20% aq KHCO₃ and stirred for 1 minute, then treated with a solution of tert-butyl carbazate (21.12 g, 160 mmol) in ethyl acetate (75 ml) and heated to reflux for 4 hours. The layers were separated, the organic phase washed with brine (30 ml), dried over MgSO₄ and evaporated to a solid which was boiled in cyclohexane (100 ml) for 1 minute, cooled and filtered to yield tert-butyl 2-{2-[(2,3-difluorophenyl)amino]-2-oxoethyl}hydrazinecarboxylate.

Yield: 20.68 g (86%); white crystals.

¹H NMR (DMSO-d6): δ=9.97 (br s, 1H), 8.46 (br s, 1H), 7.79 (m, 1H), 7.16 (m, 2H), 5.36 (m, 1H); 3.29 (d, J=3.7 Hz), 1.36 (s, 9H).

MS: m/z=301 (M⁻).

Anal. Calcd. for C₁₃H₁₇F₂N₃O₃: C, 54.72; H, 4.92; N, 22.79. Found: C, 51.9; H, 5.66; N, 13.9.

### {2-[(2,3-difluorophenyl)amino]-2-oxoethyl}hydrazinium dimethanesulfonate

A solution of tert-butyl 2-{2-[(2,3-difluorophenyl)amino]-2-oxoethyl}hydrazinecarboxylate (3.01 g, 10 mmol) in ethyl acetate (70 ml) was treated dropwise over 2 minutes with methanesulfonic acid (1.92 g, 20 mmol). The resultant slurry was heated to reflux for 100 minutes, cooled, the thick precipitate filtered off, washed with ethyl acetate (100 ml) and diethyl ether (100 ml) and air-dried.

Yield: 3.87 g (98%); white powder.

¹H-NMR (DMSO-d6): δ=10.20 (s, 1H), 7.70 (m, 1H), 7.21 (m, 2H), 3.82 (s, 2H); 2.41 (s, 6H).

MS: m/z=201 (M⁻).

Anal. Calcd. for C₈H₉F₂N₃O.C₂H₈O₆S₂: C, 30.53; H, 4.36; N, 10.68. Found: C, 30.2; H, 4.37; N, 10.6.

### N-(2,3-difluorophenyl)-2-(4-{[(dimethylamino)methylene]amino}-1H-pyrazol-1-yl)acetamide

A solution of N-(3-(dimethylamino)-2-{[(dimethylamino)methylene]amino}prop-2-en-1-ylidene)-N-methylmethanaminium hydrogen monohexafluorophosphate (3.76 g, 11 mmol) in dry pyridine (20 ml) was cooled to −25° C. under nitrogen and treated with a solution of {2-[(2,3-difluorophenyl)amino]-2-oxoethyl}hydrazinium dimethanesulfonate (3.93 g, 10 mmol) in dry methanol (30 ml) and pyridine (30 ml), over 5 minutes at −15 to −30° C. To the resultant slurry, a solution of 30% w/v methanolic sodium methoxide (5.66 g, 31 mmol) in methanol (5 ml) was then added over 5 minutes at −27 to −30° C. The resultant yellow solution was allowed to warm to room temperature over 1 hour and was then heated to 60° C. for 15 minutes, cooled, treated with acetic acid (3 ml) and evaporated to a black residue which was azeotroped with toluene (2×50 ml) to remove pyridine, then extracted twice with boiling water (125 ml, 50 ml) and the aqueous solution filtered through celite. The filtrate was basified to pH 7-8 with 20% aq KHCO₃, extracted with ethyl acetate (3×100 ml), the combined organic solution extracted with brine (3×20 ml), dried over MgSO₄ and evaporated to a solid, which was a 90% pure mixture of N-(2,3-difluorophenyl)-2-(4-{[(dimethylamino)methylene]amino}-1H-pyrazol-1-yl)acetamide and N-(2,3-difluorophenyl)-2-[4-(formylamino)-1H-pyrazol-1-yl]acetamide. An analytically pure sample was obtained by trituration with a 1:1 mixture of ethyl acetate and tert-butyl methyl ether followed by recrystallization from ethyl acetate.

Yield (as mixture): 2.62 g (71% approx); analytical sample off-white needles.

¹H NMR (DMSO-d6): δ=10.15 (s, 1H); 7.78 (s, 1H); 7.71 (m, 1H); 7.44 (s, 1H); 7.29 (s, 1H); 7.18 (m, 1H); 4.98 (s, 2H); 2.89 (s, 6H).

MS: m/z=308 (M⁺).

Anal. Calcd. for C₁₄H₁₅F₂N₅O: C, 54.72; H, 4.92; N, 22.79. Found: C, 54.5; H, 4.83; N, 22.6.

The same product was also made as follows:

N,N-dimethyl-N′-1H-pyrazol-4-ylimidoformamide (152 mg, 1.1 mmol) in dimethylformamide (0.5 mL) and 1,4-dioxane (0.5 mL) was treated with sodium hydride 60% oil dispersion (40 mg, 1.0 mmol) and stirred for 20 minutes. The brown solution was cooled to 5° C. and a solution of 2-bromo-N-(2,3-difluorophenyl)acetamide (251 mg, 1.0 mmol) in 1,4-dioxane (0.5 mL) was added dropwise. The solution was stirred at room temperature for 30 minutes, then treated with acetic acid (3 mL) and evaporated under a nitrogen stream. The residue was purified by chromatography on silica, eluting with dichloromethane:methanol 100:6 to 100:20 to give the product as a brown gum. Yield (with impurities) 40 mg (13%).

¹H NMR (DMSO-d6): δ=10.18 (s, 1H); 7.86 (s, 1H); 7.71 (m, 1H); 7.49 (s, 1H); 7.33 (s, 1H); 7.19 (m, 1H); 5.00 (s, 2H); 2.91 (s, 6H).

MS: m/z=308 (M⁺).

### 2-(4-Amino-1H-pyrazol-1-yl)-N-(2,3-difluorophenyl)acetamide

N-(2,3-difluorophenyl)-2-(4-{[(dimethylamino)methylene]amino}-1H-pyrazol-1-yl)acetamide (2.44 g, 8 mmol) was dissolved in a boiling mixture of water (50 ml) and n-propanol (10 ml), allowed to cool to 50° C. and treated dropwise with conc. aq NH₄OH (1.33 ml, 24 mmol). The solution was heated to reflux for 20 minutes, allowed to cool to 70° C. and acidified to pH 7 with SM aq H₂SO₄ (c. 0.5 ml) followed by another 4.8 ml (24 mmol) of the same acid. After 90 minutes at 70° C., the solution was cooled, basified to pH 7.5 with aq. NH₄OH and evaporated to 60 ml. The product was extracted into ethyl acetate (3×50 ml), the organic phase dried over MgSO₄ and evaporated to an oil which was purified by chromatography on silica gel eluting with CH₂Cl₂:methanol 100:3 to 100:8.

Yield: 762 mg (45%); pale pink solid.

¹H NMR (DMSO-d6): δ=10.07 (br s, 1H); 7.71 (m, 1H); 7.18 (m, 2H); 7.09 (s, 1H); 6.98 (s, 1H); 4.91 (s, 2H); 3.86 (br s, 2H).

MS: m/z=252 (M⁺).

Anal. Calcd. for C₁₁H₁₀F₂N₄O: C, 52.38; H, 4.00; N, 22.21. Found: C, 52.2; H, 3.86; N, 22.0.

A process of the invention has been used in the preparation of N,N-dimethyl-N′-1H-pyrazol-4-ylimidoformamide.

### N,N-Dimethyl-N′-1H-pyrazol-4-ylimidoformamide

A solution of N-((2Z)-3-(dimethylamino)-2-{[(1E)-(dimethylamino)methylene]amino}prop-2-en-1-ylidene)-N-methylmethanaminium hexafluorophosphate (3.76 g, 11 mmol) in dry pyridine (20 ml) at −25° C. was stirred under nitrogen while adding a mixture of 30% w/v methanolic NaOMe (6.30 g, 35 mmol) and 1M hydrazine in THF (10 ml, 10 mmol) in anhydrous methanol (5 ml), over 5 minutes at −20 to −30° C. The resultant solution was allowed to warm to room temperature over 30 minutes and was then heated to 60° C. for 20 m, cooled, treated with acetic acid (3 ml) and evaporated to a residue which was azeotroped with toluene (2×50 ml) to remove pyridine. The residue was taken into dichloromethane:methanol:aqueous ammonium hydroxide 100:25:2 (100 ml) and filtered through 100 g silica (sinter). Evaporation gave a residue that was purified by chromatograpy on silica eluting with dichloromethane containing 3% of a 10:1 mixture of methanol and aqueous ammonia, increasing the gradient to 15%. N,N-Dimethyl-N′-1H-pyrazol-4-ylimidoformamide was obtained as an oil, 195 mg (14%).

¹H NMR (DMSO-d6): δ=7.74 (s, 1H), 7.32 (s, 1H), 2.86 (s, 6H).

MS: m/z=138 (M⁺).

1. A process comprising the reaction of a compound of formula (G) 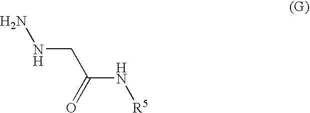
with a compound of formula (F) 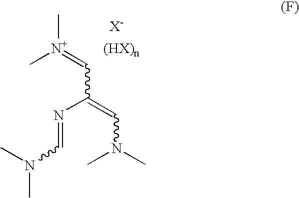
in the presence of a base; wherein X is PF₆ or BF₄; n is 0 or 1; and R⁵ is optionally substituted aryl or heteroaryl;
to yield a compound of formula (H):

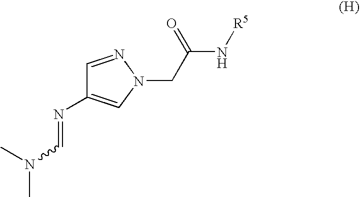

2. A process according to claim 1 further comprising the hydrolysis of a compound of formula (H) to yield a compound of formula (I).

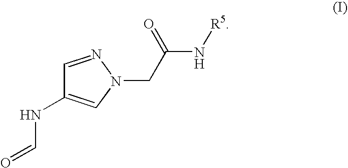

3. A process according to claim 2 further comprising deformylation of a compound of formula (I) to yield a compound of formula (C).

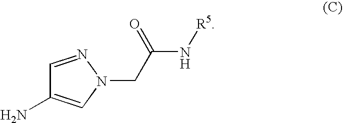

4. A process according to claim 1 further comprising converting a compound of formula (H) to a compound of formula (C) under acidic conditions.

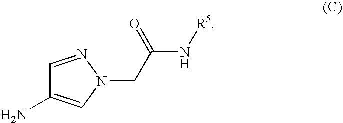

5. A process comprising the hydrolysis of a compound of formula (H): 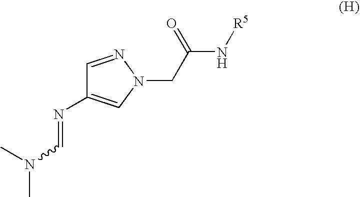
to yield a compound of formula (I) 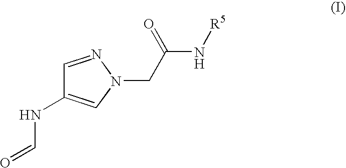
wherein R⁵ is optionally substituted aryl or heteroaryl.

6. A process comprising deformylation of a compound of formula (I): 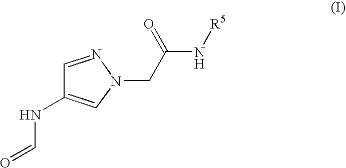
to yield a compound of formula (C): 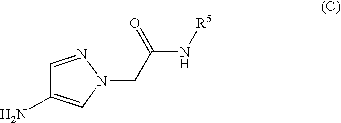
wherein R⁵ is optionally substituted aryl or heteroaryl. H₂N (C)

7. A process comprising converting a compound of formula (H); 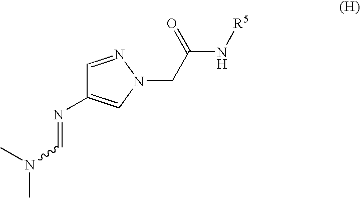
into a compound of formula (C): 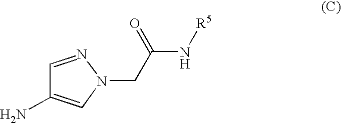
under acidic conditions wherein R⁵ is optionally substituted aryl or heteroaryl. H₂N (C)

8. A process according to claim 1 wherein R⁵ is aryl optionally substituted by 1 or 2 halo.

9. A compound of formula (G) 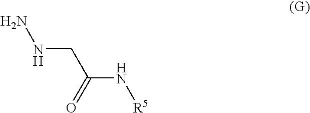
wherein R⁵ is aryl or R⁵ is aryl or heteroaryl substituted by 1 or 2 halo; or a salt thereof.

10. A compound according to claim 9 or a salt thereof selected from {2-[(2,3-difluorophenyl)amino]-2-oxoethyl}hydrazine and {2-[(3-fluorophenyl)amino]-2-oxoethyl}hydrazine.

11. A compound of formula (H) 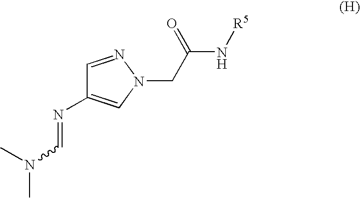
wherein R⁵ is optionally substituted aryl or heteroaryl; or a salt thereof.

12. A compound of formula (I) 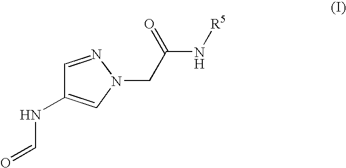
wherein R⁵ is optionally substituted aryl or heteroaryl; or a salt thereof.

13. A process comprising the reaction of a compound of formula (F): 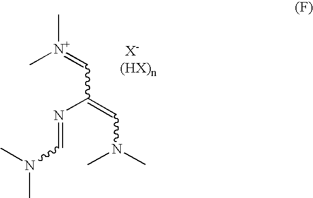
with hydrazine to yield a compound of formula (K)

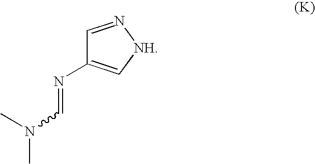

14. A compound of formula (K) or a salt thereof:

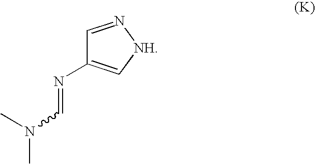

15. A process comprising the reaction of a compound of formula (K)
with a compound of formula (J) 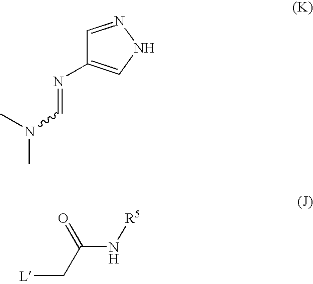
wherein L′ is a leaving group and R⁵ is optionally substituted aryl or heteroaryl;
to yield a compound of formula (H).

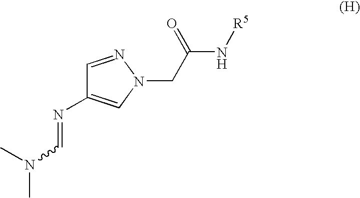

16. A process comprising the hydrolysis of a compound of formula (K) 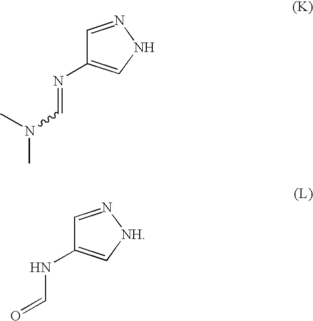
to yield a compound of formula (L).

17. A compound of formula (L) or a salt thereof.

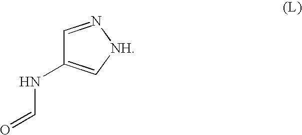

18. A process comprising deformylation of a compound of formula (L) 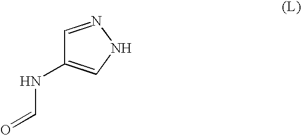
to yield 4-aminopyrazole.

19. A process comprising the conversion of a compound of formula (K): 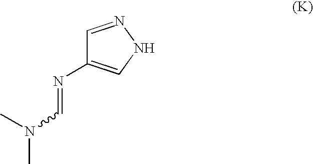
to 4-aminopyrazole under acidic conditions.
